(19) United States
(12) Patent Application Publication
(10) Pub. No.: US 2016/0051392 A1
(43) Pub. Date: Feb. 25, 2016

(54) LOWER BACK PAIN TREATMENT AND SPINAL PROTECTION DEVICE

(71) Applicants: Rosalie Dudkiewicz, West Orange, NJ (US); Dariusz Dudkiewicz, West Orange, NJ (US)

(72) Inventors: Rosalie Dudkiewicz, West Orange, NJ (US); Dariusz Dudkiewicz, West Orange, NJ (US)

(21) Appl. No.: 14/935,530

(22) Filed: Nov. 9, 2015

Related U.S. Application Data
(63) Continuation-in-part of application No. 14/615,841, filed on Feb. 6, 2015.
(60) Provisional application No. 62/122,467, filed on Oct. 22, 2014.
(60) Provisional application No. 61/997,154, filed on May 27, 2014.

(51) Int. Cl. A61F 5/34; A61F 5/02

(52) U.S. Cl. CPC A61F 5/34; A61F 5/024; A61F 5/028

(57) ABSTRACT

A medical device is provided. Here, the medical device has a back panel, having a top surface and a bottom surface; a plurality of inflatable pads attached to this top surface, where the plurality of inflatable pads features an outer wall having an inner and outer surface, a rigid subwall proximate to said inner surface, a diaphragm, having a volume, with at least one tube fluidly connected thereto; a connection valve, fluidly connected to the tube. The medical device the incidental contact of a given object with a user's spine, and prevents the compression of this user's spine.

### CLAIM OF PRIORITY

This application is a continuation-in-part of U.S. patent application Ser. No. 14/615,841, filed on Feb. 6, 2015, entitled “LOWER BACK PAIN TEATMENT AND SPINAL PROTECTION DEVICE”, which claims priority from U.S. Provisional Patent Application No. 62/122,467, filed on Oct. 22, 2014 and U.S. Provisional patent application No. 61/997,154, filed on May 27, 2014 and the contents of which are hereby incorporated by reference.’

### FIELD OF THE EMBODIMENTS

The invention and its embodiments relate to medical devices, namely a device that is capable of alleviating lower back pain. In particular, the present invention and its embodiments relate to a device that prevents the lumbar spine process and its peripheral nerves from being touched or compressed.

### BACKGROUND OF THE EMBODIMENTS

There are about 150-200 million adults that suffer from low back pain in United States, and it is the second most common reason people seek a doctor. This health problem results in about 2.4 million Americans chronically disabled and temporarily on disability. Lower back pain affects the spine's stability, flexibility, and strength as result cause pain, discomfort and stiffness during daily living activities such as sitting, driving or sleeping. A great majority of back pain experienced by the general population occurs in the lower portion of the back generally referred to as the lumbar area or the spinal segments L-3 through S-1 specifically. In order to comprehend the most causes of low back pain, it is essential to understand the functioning of the spinal column. The purpose of the human spinal column is to support and protects the body. The most common area of low back pain is at the spinal process segment L-3 to S-1. The spine process is composed of twenty four individual vertebrae. Each vertebra is attached and support by cartilages, muscles and ligaments to allow flexibility to bend over, and to rotate the torso. Each vertebra is connected by an intervertebral disc that functioning to provide cushion, and prevent compression to the peripheral spinal nerves. Whenever there is a shift of the vertebrae from its normal position caused by herniated discs, spinal stenosis, fractures, osteoarthritis or injury, it presses against the spinal nerves, and as a result it stimulates pain during daily living activities such as a sitting, sleeping, driving or bending. Likewise, low pain back is also caused by, the shifting of the 2 vertebrae caused by unequal pressure of the muscles supporting the spine column. This can occur due to overuse or injury of the muscles or ligaments, uneven muscular stress, emotional tension. The majority of low back pain is managed with conservative treatment such as rest, analgesic, anti-inflammatory medications in conjunction with physical therapy to strength the back muscles to support the spine column to relief pain or eliminate it. Another conventional treatment used to address back pain due to injury or non-injury is applying pressure to the area with orthopedic belt devices.

### Review of Related Technology:

U.S. Pat. No. 8,226,587 pertains to a spinal support and brace device configured to support the lower back and that anchors on portion for the pelvis and are configured to support and align one or more lumber vertebrae.

International Patent Application No.: WO2007027573 pertains to a lumbar lordosis correction brace comprised of a lower portion, adapted to be comfortably affixed to the lower truck of a patient, and upper portion, adapted for comfortably encircling the upper trunk of a patient such that the two portions work together to provide uniform pressure against a patient's spine. This patent application also provides for the ability to displace, in a controlled manner, to provide additional support of the lumbar.

U.S. Pat. No. 5,405,313 pertains to an adjustable back support comprising a pelvic girdle that has been adapted to fit snugly around a user's waist, a pair of hip crutches adapted to be vertically disposed on each side of a user's body, wherein each hip crutch extends upwardly at a small forward angle between the pelvic girdle and the corresponding armpit.

US Patent Publication No.: 2010/0168630 pertains to a rigid lumbar compression piece, that includes first and second, separate, opposing and mating front attachment panels; and a separate, laterally adjustable, rigid lumbar compression piece configured for positioning only at the rear of a wearer, wherein pulling of a cord causes the brace both circumferentially to tighten and to concentrate compression and pressure of the separate, laterally adjustable, rigid lumbar compression piece pushed directly against a spinal region of a wearer's back with the aid of a number of apertures through which the cord or cords pass.

US Patent Publication No.: 2013/0090585 pertains to a neuromuscular training device for enhancing the alignment of the upper torso of a user. The device is preferably configured specifically to provide scapula retraction and stabilization while providing stated and dynamic neuromuscular training.

U.S. Pat. No. 5,334,134 pertains to a lumbosacral back support member which is releasably secured to the user by a stabilizing belt. Generally, this device is used over a user's clothes. This stabilizing belt may be inserted into the belt loops of a user's pants, attached to these belt loops, or engaged within the narrow portions of the user's waist.

U.S. Pat. No. 5,399,150 pertains to a lumbosacral back support system with interchangeably and postionally-adjustable lumbosacral orthopedic support members for supporting the spine of a user during physical activity, athletics activities, or for use by industrial workers.

U.S. Pat. No. 8,012,113 pertains to a spinal brace that includes a flexible air injectable band configured to be disposed about a torso of a user and to provide traction to a spine of the user. The device also comprises at least one support panel configured to provide support by compression to at least one region of a user's torso.

US Patent Publication No.: 2014/0221892 pertains to a maternity support belt that includes a curved support edge which matches the female pelvic curve, to support a gravid abdomen. The maternity belt includes a lumbosacral supporting section that applies a compressive force against the lumbosacral portion of the wearer's back, and a pair of gravid abdominal support sections on opposite lateral sides of the lumbosacral supporting section and contiguous thereto. The gravid abdominal support sections adjustably overlap and connect to each other on the wearer's front side.

U.S. Design Pat. No. D657063 pertains to a back support.

Various devices are known in the art. However, their structure and means of operation are substantially different from the present invention. Such devices fail to provide a device that can be easily operated through medical gloves and that provide a tool that can be used on a wider array of hemorrhoidal tissue. At least one embodiment of this invention is presented in the drawings below and will be described in more detail herein.

### SUMMARY OF THE EMBODIMENTS

The present invention provides for a medical device, comprising: a back panel, having a top surface and a bottom surface; a plurality of inflatable pads attached to said top surface, said plurality of inflatable pads comprising an outer wall having an inner and outer surface, a rigid subwall proximate to said inner surface, a diaphragm, having a volume, with at least one tube fluidly connected thereto; a connection valve, fluidly connected to said at least one tube; wherein said medical device prevents the incidental contact of a given object with a user's spine, and wherein said medical device prevents the compression of said user's spine. In some embodiments, the present invention further comprises expandable material located between said outer wall and said top surface. Additionally, the present invention may feature a plurality of vents, having a first opening on said outer wall and a second opening on said bottom surface of said back panel wherein said first opening and said second opening are in fluid communication with each other. Preferably, the back panel is substantially rigid and the device promotes proper posture by properly aligning a user's spine. In some embodiments, the plurality of inflatable pads is equipped with a hollow center portion, sized to accommodate the growth of a cyst or tumor of a user proximate to said plurality of inflatable pads and preferably said plurality of inflatable pads are semicylindrical. In many embodiments, said connection valve is used to inflate and deflate said diaphragm and in others, said plurality of inflatable pads is removably detached to said top surface. The device of the present invention may be constructed out of a material that is imbued with an antimicrobial agent, such as silver nanoparticles. The present invention may have a connection vale with a closed-bias and may have the plurality of inflatable pads be attached via hook and loop fasteners.

The present invention is a device to help individuals suffering from acute or chronic lower back pain to perform their daily living activities such as sitting, sleeping or driving without or with less pain. The invention also allows individuals recovering from back surgery or back injury to recovery fast. It accomplishes it by the following: It protects the incision site (stitches) at the spine from been compressed and contact with any object during sitting (chair or sofa) or sleeping (mattress), driving or riding in a car; air can flow freely to the surgical site, as a result promote healing; the individual is able to sit or lie down in a back resting position without having pressure against the incision site. In another embodiment of the invention, the cushion pads may be carved lateral to the spine to accommodate rods or others device placed in the back during back surgery. This feature prevents the rods or devices from being compressed during sleeping/sitting or driving which can be uncomfortable to the wearer. In addition, the device can be continuing used for long period of time post-surgery in people who has to return to work and they perform desk work such as secretary or job as truck driver. It also promotes good posture which in turn prevents future low back pain. The present invention's function is to reduce or eliminate pain by preventing the lumbar spine process and its peripheral nerves from being touched or compressed by any object during sitting, sleeping, or driving. The device accomplishes this by helping the erector spinae muscles to support the spine process to be in proper alignment, and by preventing the spine from touch any object that has potential to compress it, and stimulate pain. Another feature of the device is that the front embodiment spandex fabric gently compresses the abdominal cavity, and intra-abdominal pressure is increased, and as a result the lumbar is supported and corrected. This mechanism relief pain and promote proper posture. Moreover, it can be used comfortable 24/7 hours week discreet under the wearer garment. The inside back part is enclosed by an envelope case appropriate designed for female, male and pregnant woman. In addition, the Low Back Pain solution device has a tail end piece that fits well inside the user's underwear, and it is hidden well that only the individual knows that she/he is wearing it, as a result it ease the user's pain, without feeling embarrassed to use it. In general, the present invention succeeds in conferring the following, and other not mentioned, benefits and objectives.

It is an object of the present invention to provide a means for relieving back support.

It is an object of the present invention to provide a medical device.

It is an object of the present invention to provide a means for removing pressure from a user's lumbar.

It is an object of the present invention to provide a device that supports the erector spinae muscles.

It is an object of the present invention to provide a means for protecting surgical incisions along the spine.

### BRIEF DESCRIPTION OF THE DRAWINGS

FIG. 1 is a top perspective view of an embodiment of the present invention.

FIG. 2 is a side view of an embodiment of the present invention.

FIG. 3 is a bottom view of an embodiment of the present invention.

FIG. 4 is an alternative top view of an embodiment of the present invention, with the inflatable pads of the present invention being removed.

FIG. 5 is a rear perspective view of an embodiment of the inflatable pads of the present invention.

FIG. 6 is a perspective view of an alternative embodiment of the inflatable pads of the present invention.

FIG. 7 is a perspective x-ray view of an embodiment of the present invention.

FIG. 8 is a side cross-sectional view of an embodiment of the inflatable pad of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Low Back Pain solution device features two main components: the outside envelope and the back section. These components can be constructed from, by non-limiting example ethyl vinyl acetate (EVA) or nitrile butadiene rubber foam. In one embodiment, the back section is a single piece shaped similarly to a human buttocks. In a preferred embodiment, the back section features two cushion pads, as well as a cavity. Preferably, this cavity will be 6 centimeters wide. The purpose of this cavity is to protect the spinal column and peripheral nerves from being touched or compressed by any object. Objects that can frequently cause such touching or compression include chairs, sofas, beds or car seats. This function also prevents pressure against incision site of post-spine surgery individual or individual with injured back. It allows the individual to perform daily activities such as sleeping, sitting or driving.

Further, any spinal incision is protected from being touched by any object during the above activities, which makes a user's healing process fast and effective. In turn, this makes the recovery period shorter. Another significant feature of this device is the two soft yet firm cushioned pads. Preferably, these pads have rectangular base with a rounded top, however, this does not mean that other shapes would not be suitable. This rounded top lies on the erector spinae muscles allowing the spine process and its nerves to be free from contact due to any object. In a preferred embodiment, these pads are aligned parallel to a user's lower spine processes and erector spinae musculature. These pads hold the muscles in place to support the spine; allowing good blood flow to the area, and it prevents the muscles to fatigue, which also promotes healing.

The present invention also facilitates the correction of curvature and extension of a user's spine. This facilitation results in easing a user's back pain while helping them maintain proper posture. Additionally, the outside part has the purpose to enclose the back piece, as well it front is made of spandex material that helps support the abdominal muscles, a result promote proper posture and support the spine. In yet another preferred embodiment, the cavity has preferred dimensions of 7 cm wide while being and 2.5 cm deep, having a layer of 2.4 mm sheet of EVA and NBR foam. In one embodiment, the back section is enclosed by a comfortable non-spandex and washable fabric, but this should not be construed as the invention being limited to these fabrics.

The present invention provides for a spinal brace with air inflatable protrusions to shelter the spine (nerves, joints and disks) from been in contact with any object during, sitting, standing, or sleeping.

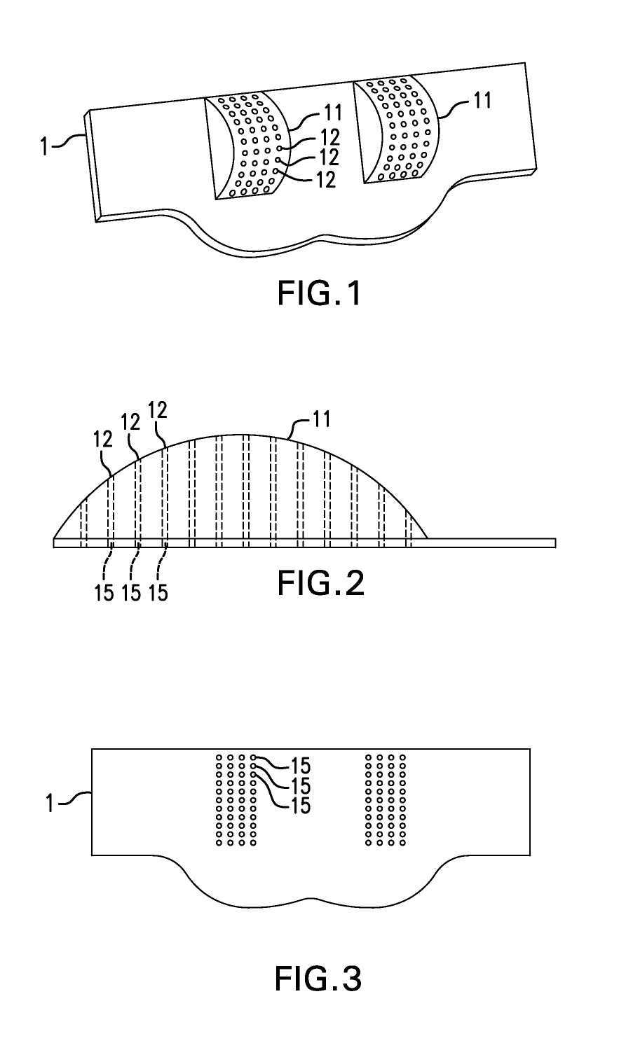

Referring to FIG. 1, an embodiment of the present invention is shown. This embodiment features back panel 1, plurality of inflatable pads 11, and plurality of vents 12. Preferably plurality of inflatable pads 11 will have a semicylindrical shape when inflated, although other shapes are contemplated by the present invention. The present invention operates by securing the device to a user's lower back, with the user's spine being located in between plurality of inflatable pads 11. With plurality of inflatable pads 11 making contact with the area flanking a user's spine, compression of that spinal area is made impossible due to the support provided by plurality of inflatable pads 11. Further, due to the preferred rigidity of back panel 1, should a user bump up against an object inadvertently, their spine will still be shielded by the present invention. Plurality of vents 12 is a desirable feature of this embodiment as many of the material suitable for use in constructing plurality of inflatable pads 11 are plastics that do not breathe at all. As such, the inclusion of plurality of vents 12 will prevent a user from overheating while using the device of the present invention.

FIG. 2 shows a side view of an embodiment of the present invention. This view shows the relationship between plurality of vents 12, second opening 15, and plurality of inflatable pads 11. Plurality of vents 12 operates by allowing any fluid to freely flow from one opening disposed on the back surface of back panel 1, and the other opening disposed on the top of plurality of inflatable pads 11. This fluid is preferably air, but a cooling liquid could also be transported through plurality of vents 12.

Referring to FIG. 3, a bottom view of an embodiment of the present invention is provided. Here one can see second opening 15 of the plurality of vents 12 (See FIG. 2). As evidenced by this figure, second opening 15 is on the bottom surface of the rigid back panel of the present invention. This allows air from the ambient environment to be used to cool a user of the device of the present invention.

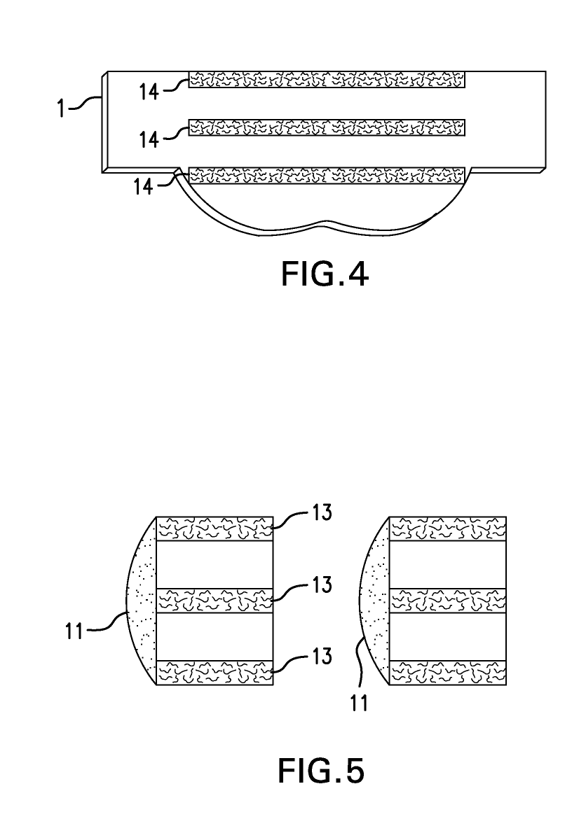

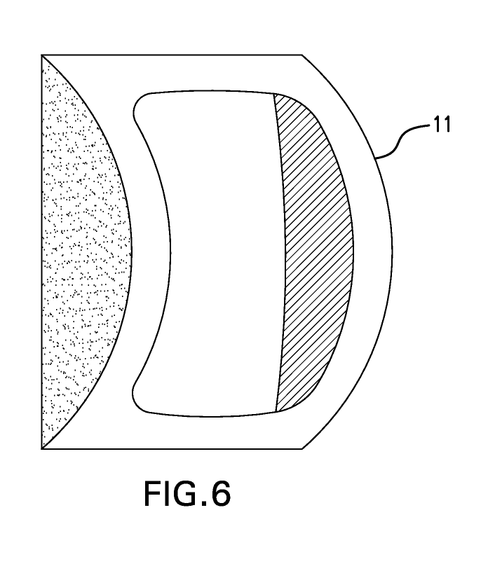

Turning to FIG. 4, an alternative top view of an embodiment of the present invention, with the inflatable pads of the present invention being removed. In this embodiment back panel 1 is disposed with a plurality of loop fasteners 14. In other embodiments, plurality of hook fasteners 13 (See FIG. 5) are disposed on back panel 1 in lieu of plurality of loop fasteners 14. Here, plurality of loop fasteners 14 provide for the removable attachment of an embodiment of inflatable pad 11 equipped with plurality of hook fasteners 13. One benefit of this embodiment is that the device may be more easily stored when inflatable pads 11 (See FIG. 5) are detached from back panel 1. Another benefit is the ability to adjust the distance between inflatable pads 11 (See FIG. 5) when attached to back panel 1. This is beneficial because two given users may have spines with different widths, or may have other ailments that would mean that more widely spaced inflatable pads 11 (See FIG. 5) would be preferential.

FIG. 5 is a rear perspective view of an embodiment of the inflatable pads of the present invention. This embodiment is intended to interface with the embodiment shown in FIG. 4. Here, inflatable pad 11 is equipped with plurality of hook fasteners 13. Much like the embodiment in FIG. 4, plurality of hook fasteners 13 may be replaced with plurality of loop fasteners 14. As noted above, this configuration provides for a more customizable experience while wearing the device of the present invention.

Referring to FIG. 6, a perspective view of an alternative embodiment of the inflatable pads of the present invention. This embodiment of inflatable pad 11 features a hollow portion in the center of the pad. That is, inflatable pad 11 is still inflatable, and in some embodiments removably attachable, but now when inflated a cavity exists in the center of the pad. This is to accommodate a user who has grown a cyst, has an implanted medical device, or has some other ailment that is sensitive to touch. This embodiment allows the touch-sensitized area to be isolated, while still providing the back support and compression prevention that the other embodiments of inflatable pads 11 provides, which is particularly useful for sleeping.

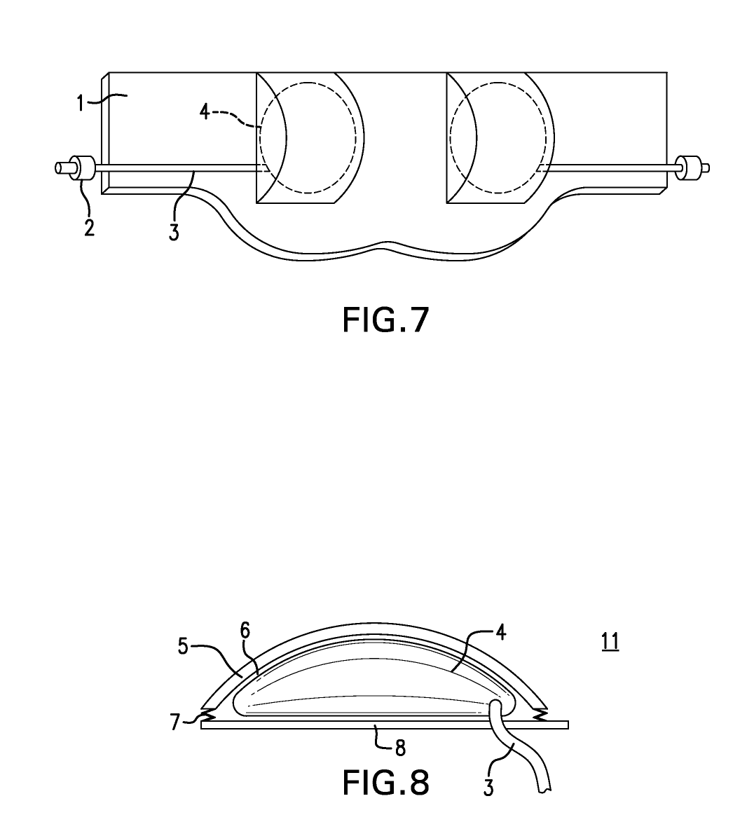

FIG. 7 shows a perspective x-ray view of an embodiment of the present invention. Here, the present invention features back panel 1, connectional valve 2, at least one tube 3, and diaphragm 4. As can be seen, connection valve 2, at least one tube 3, and diaphragm 4 are in fluid connection with each other. A user will force air through connection valve 2, through at least one tube 3, and into diaphragm 4 such that the diaphragm will expand, inflating inflatable pad 11. A user may use an air pump, or they may merely blow into the connection valve to achieve this result. Preferably, connection valve 2 will have a closed-bias. That is, it will remain close unless the requisite amount of force is exerted in the valve, or the valve is manipulated such that the ambient environment becomes in fluid connection with at least one tube 3, and diaphragm 4. In an alternative embodiment, connection valve 2 has a releasing valve that remains closed during the inflation process, and it is open during the deflation process.

Referring to FIG. 8, a side cross-sectional view of an embodiment of the inflatable pad of the present invention. This view shows at least one tube 3, diaphragm 4, outer wall 5, rigid subwall 6, expandable material 7, and rigid wall 8. Here, diaphragm 4 can be inflated to a desired height and/or pressure. This functionality allows for improved comfort in a user experienced lower back pain, regardless of whether the user is standing, sitting, or laying down. Inflatable pad 11 is equipped with a diaphragm 4 which is in fluid connection with at least one tube 3 ending with connection valve 2. In a preferred embodiment, to accommodate for and change in height during inflation/deflation of inflatable pad 11, and also to prevent bending of outer wall 5, outer wall 5 is connection to rigid wall 8, via expandable material 7. Expandable material 7 add some flexibility in this mechanism such that the structural integrity of inflatable pad 11 will be persevered throughout a multitude of inflation/deflation cycles.

In another embodiment of the present invention, the invention may be adapted and configured to support other bones of the body, including but not limited to supporting the bones of the arms and legs. In another embodiment of the present invention, the pads that create the channel or cavity where the user's spine fits into may be a unitary component. In another embodiment, the present invention may be worn as an outer garment such as a corset. In another embodiment, the present invention may be completely waterproof. In another embodiment, the interior pads may be removable, adjustable and detachable. In another embodiment of the invention, a user may insert the pads into the device and the pads are customizable and may be placed to fit any user's spine or measurements. In another embodiment, the pads can be of any material and of various thicknesses to confirm to a user's body. In another embodiment, the present invention may contain heating or cooling pads within the pads or within the channel or cavity.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. In some embodiments, the device of the present invention is constructed, in whole or in part out of a material containing an antimicrobial agent, such as silver nanoparticles.

While the disclosure refers to exemplary embodiments, it will be understood by those skilled in the art that various changes may be made and equivalents may be substituted for elements thereof without departing from the scope of the disclosure. In addition, many modifications will be appreciated by those skilled in the art to adapt a particular instrument, situation or material to the teachings of the disclosure without departing from the spirit thereof. Therefore, it is intended that the disclosure not be limited to the particular embodiments disclosed.

What is claimed is:

1. A medical device, comprising:
a back panel, having a top surface and a bottom surface;
a plurality of inflatable pads attached to said top surface,
said plurality of inflatable pads comprising an outer wall having an inner and outer surface, a rigid subwall proximate to said inner surface, a diaphragm, having a volume, with at least one tube fluidly connected thereto;
a connection valve, fluidly connected to said at least one tube;
wherein said medical device prevents the incidental contact of a given object with a user's spine, and
wherein said medical device prevents the compression of said user's spine.

2. The medical device of claim 1, said inflatable pad further comprising expandable material located between said outer wall and said top surface.

3. The medical device of claim 1, further comprising:
a plurality of vents, having a first opening on said outer wall and a second opening on said bottom surface of said back panel,
wherein said first opening and said second opening are in fluid communication with each other.

4. The medical device of claim 1, wherein said back panel is substantially rigid.

5. The medical device of claim 1, wherein said device promotes proper posture by properly aligning a user's spine.

6. The medical device of claim 1, wherein said plurality of inflatable pads is equipped with a hollow center portion, sized to accommodate the growth of a cyst or tumor of a user proximate to said plurality of inflatable pads.

7. The medical device of claim 1, wherein said plurality of inflatable pads are semicylindrical.

8. The medical device of claim 1, wherein said plurality of inflatable pads may be disposed on multiple areas of said top surface.

9. The medical device of claim 1, wherein said connection valve is used to inflate and deflate said diaphragm.

10. The medical device of claim 1, wherein said plurality of inflatable pads is removably detached to said top surface.

11. The medical device of claim 1, wherein said device is constructed out of a material that is imbued with an antimicrobial agent.

12. The medical device of claim 1, wherein said connection valve has a closed-bias.

13. The medical device of claim 10, wherein said plurality of inflatable pads are removably attached to said surface via a plurality of hook and loop fasteners.

14. The medical device of claim 11, wherein said antimicrobial agent is silver nanoparticles.

15. A device for promoting the proper curvature of a user's spine, comprising:
a back panel, having a top surface and a bottom surface;
a plurality of inflatable pads attached to said top surface,
said plurality of inflatable pads comprising an outer wall having an inner and outer surface, a rigid subwall proximate to said inner surface, a diaphragm with at least one tube fluidly connected thereto;
a connection valve, fluidly connected to said at least one tube;
wherein said medical device prevents the incidental contact of a given object with a user's spine, and
wherein said medical device prevents the compression of said user's spine.
